SEQUENCE LISTING (11 sequences)

SEQ ID NO: 1 - DNA, 20 bp, Artificial Sequence
  misc_feature: Description of Artificial Sequence  Primer
ggaagcttgcctgtactggg

SEQ ID NO: 2 - DNA, 24 bp, Artificial Sequence
  misc_feature: Description of Artificial Sequence  Primer
accctcgagagaccagttcgaagg

SEQ ID NO: 3 - DNA, 21 bp, Artificial Sequence
  misc_feature: Description of Artificial Sequence  Primer
ggaagcttgacagccgcctag

SEQ ID NO: 4 - DNA, 19 bp, Artificial Sequence
  misc_feature: Description of Artificial Sequence  Primer
tcggagttatttcgaacgg

SEQ ID NO: 5 - DNA, 19 bp, Artificial Sequence
  misc_feature: Description of Artificial Sequence  Primer
ggaagcttgccttgagtgc

SEQ ID NO: 6 - DNA, 22 bp, Artificial Sequence
  misc_feature: Description of Artificial Sequence  Primer
ccgctccccgccgcttcgaagg

SEQ ID NO: 7 - DNA, 22 bp, Artificial Sequence
  misc_feature: Description of Artificial Sequence  Primer
ggaagcttggactcggcttgct

SEQ ID NO: 8 - DNA, 20 bp, Artificial Sequence
  misc_feature: Description of Artificial Sequence  Primer
tgatcgcctccgttcgaagg

SEQ ID NO: 9 - DNA, 23 bp, Artificial Sequence
  misc_feature: Description of Artificial Sequence  Primer
ggaagcttgcgaaagggaaacca

SEQ ID NO: 10 - DNA, 19 bp, Artificial Sequence
  misc_feature: Description of Artificial Sequence  Primer
tctgtggttccttcgaagg

SEQ ID NO: 11 - DNA, 1200 bp, Human Immunodeficiency Virus
tggaagggctaattcactcccaacgaagacaagatatccttgatctgtggatctaccacacacaaggctacttccctgattagcagaactacacaccagggccagggatcagatatccactgacctttggatggtgctacaagctagtaccagttgagccagagaagttagaagaagccaacaaaggagagaacaccagcttgttacaccctgtgagcctgcatggaatggatgacccggagagagaagtgttagagtggaggtttgacagccgcctagcatttcatcacatggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaac

(19) United States
(12) Patent Application Publication
(10) Pub. No.: US 2004/0101821 A1
(43) Pub. Date: May 27, 2004

(54) Antiviral antisense therapy

(76) Inventors: Andrew Lever, Cambridge (GB); David Robert Chadwick, Cambridge (GB)

Correspondence Address: NIXON & VANDERHYE, PC, 1100 N GLEBE ROAD, 8TH FLOOR, ARLINGTON, VA, 22201-4714, US

(21) Appl. No.: 10/296,262

(22) PCT Filed: May 23, 2001
(86) PCT No.: PCT/GB01/02310

(30) Foreign Application Priority Data
May 23, 2000 (GB) 0012497.4

(51) Int. Cl. A61K 48/00; C07H 21/02; C12Q 1/70

(52) U.S. Cl. 435/5; 514/44; 536/23.72

(57) ABSTRACT

A polynucleotide which is (i) an antisense polynucleotide that binds the splice-donor/packaging signal region (SD/ψ) or the TAR region of HIV-1 RNA, or (ii) a vector polynucleotide capable of expressing (i); for use in a method of treating or preventing HIV infection.

### FIELD OF THE INVENTION

The invention relates to an antisense polynucleotide and a vector capable of expressing the antisense polynucleotide for HIV-1 therapy, particular fragments of HIV-1 RNA and a method of screening for agents able to prevent or treat HIV-1 infection.

### BACKGROUND TO THE INVENTION

The 5′ long terminal repeat (LTR) and leader regions of HIV RNA contain a number of non-coding sequences which mediate several fuctions in the viral lifecycle. Such sequences may cause the viral RNA to adopt stem-loop secondary structures. In particular the TAR region and the packaging signal of HIV RNA are capable of forming such structures.

### SUMMARY OF THE INVENTION

The inventors have shown that antisense polynucleotides which bind the TAR region or splice-donor/packaging signal (SD/ψ) region cause significant inhibition of HIV replication. They have also found that antisense polynucleotides which bind only the SD/ψ region and not the flanking primer binding site and gag coding sequences are particularly effective at causing inhibition of replication.

Accordingly the invention provides a polynucleotide which is

(i) an antisense polynucleotide that binds the splice-donor/packaging signal region (SD/ψ) or the TAR region of HIV-1 RNA, or

(ii) a vector polynucleotide capable of expressing (i);

for use in a method of treating or preventing EIV infection.

In a preferred embodiment the antisense polynucleotide binds the SD/T region of MV-1 RNA, but not the PBS or gag encoding region.

The invention also provides a method of identifying a product that is capable of treating or preventing HIV-1 infection comprising determining whether a candidate substance is capable of targeting the region bound by the antisense polynucleotide, the finding that the substance is capable of targeting the said region indicating that the substance is capable of treating or preventing HIV-1 infection.

### BRIEF DESCRIPTION OF THE DRAWINGS

FIG. 1 shows regions of HIV-1 5′ leader region and long terminal repeat (LTR) targeted by antisense RNA sequences (in italics). PBS: primer binding site; TAR: transactivation response region; ψ: packaging signal.

FIG. 2 shows RT-PCR analysis of whole cell RNA extracts from COS-1 cells transfected with antisense-expressing plasmids. Negatives refer to RT− reactions and positives to RT+ reactions (see Materials and Methods). RT reactions were primed using a reverse SP6 primer and PCR reactions using upstream primers illustrated in Table 1 and the reverse SP6 primer. Ten microlitres of PCR product was loaded onto a 1.5% agarose gel stained with ethidium bromide.

FIG. 3 shows inhibition of viral replication in Jurkat cells transfected with pcDNA3.1-antisense constructs after challenge with IRV-1 IIIB (10⁵ TCID50s). Reverse transcriptase (RT) activity was measured in cell cultures up until 17 or 21 days after challenge as described in Materials and Methods.

FIG. 4 shows proviruses derived from the vectors based on pBabePuro containing antisense cassettes in different orientations. In pBS3P the antisense sequence is expressed at the 5′ end of the puromycin gene whereas in pBS3sc the single copy cassette is placed in the reverse orientation and expressed separately to puromycin.

FIG. 5 shows inhibition of viral replication in Jurkat cells transduced with pBabePuro-based antisense vectors (italics) or transfected with pBabePuro antisense constructs (plain text) after challenge with HIV-1 IIIB (10⁵ TCID50s). Reverse transcriptase (RT) activity was measured in cell cultures until 21 days after challenge as described in Materials and Methods.

FIG. 6 shows co-transfection assays of anitisense-expressing constructs and HIV-1 gag-pol expressing vector, LψVGPH, into COS-1 cells. A. Comparison of each antisense-expressing construct with ‘vector alone’ (pcDNA3 and LψGPH) and ‘sense’-expressing vector at 3:1 ratio. B. Comparisons for constructs expressing L3, S3 and L1 sequences at variable ratios of antisense-expressing construct to vector DNA. Figures represent the amalgamation of at least three separate experiments

FIG. 7 shows RNase protection assay of cytoplasmic and virion RNA extracted from COS-1 cells co-transfected with antisense constructs (or controls) and LψGPH. Lanes 1, 8: Vector alone; Lanes 2, 9: pcL1S; Lanes 3, 10: pcL1A; Lanes 4, 11: pcL3s; Lanes 5, 12: pcL3A; Lanes 6, 13: pcS3s; Lanes 7, 14: pcS3A. 5175 nucleotide riboprobe (positions 313-830 of HXB2) was used to hybridise extracted cytoplasmic and virion RNA, and thus enable identification of unspliced genomic RNA (376 nt), singly-spliced RNA (291 nt), 3′ LTR species (141 nt) and contaminating, transfected DNA (517 nt). Lanes 15 and 16 represent yeast RNA controls with RNase digestion and without digestion respectively.

### DETAILED DESCRIPTION OF THE INVENTION

The invention provides agents that target particular regions of HIV-1 for the therapy of HIV-1 infection. In particular the invention provides a polynucleotide for use in treating or preventing infection by HIV-1 in a host. The polynucleotide may itself act as an antisense polynucleotide or may express an antisense polynucleotide. The targeting of particular regions of HIV-1 RNA causes inhibition of replication of the HIV-1 virus.

The host is generally a human, but may be any animal which can be infected by HIV-1, such as a chimpanzee or macaque. The host may have an HIV-1 infection 20 or be at risk of infection. For example the host may live in an area where HIV is endemic or may have been exposed to HIV recently. The exposure may have been by sexual contact, by an administration (such as by blood transfusion or an injection of HIV contaminated material) or by exposure to infected blood or milk from the mother of the host.

The polynucleotide is generally DNA or RNA, and is typically single or double stranded. The antisense polynucleotide may be any molecule capable of binding in a sequence specific manner, typically according to Watson-Crick base pairing. Thus the antisense polynucleotide may be any of the chemically modified polynucleotides mentioned below or may be a peptide nucleic acid (PNA). In one embodiment the antisense polynucleotide is a single stranded RNA molecule. Typically the vector polynucleotide is double stranded DNA. Where the polynucleotide is in the form of a virus vector the polynucleotide will generally be in the same form as the genome of that virus.

An antisense polynucleotide that binds a defined RNA legion is generally one that is capable of (specifically) hybridising (typically in accordance with Watson-Crick base pairing to form a duplex) to the region. Thus generally the polynucleotide is complementary, completely or partially, to the region. The polynucleotide may be the exact complement of (all) the defined region of RNA. However, absolute complementarity is not required and polynucleotides which have sufficient complementarity to form a duplex having a melting temperature of greater than 20° C., 30° C., 40° C. or 50° C. under physiological/intracellular conditions are particularly suitable.

The antisense polynucleotide is generally at least 10, for example at least 20, 40, 60, 80, 100, 200, 300, 400, 500 nucleotides in length and up to at least 700 or 1000 or more nucleotides in length.

All or part of the antisense polynucleotide may be complementary to the RNA region, and therefore all or part of the antisense polynucleotide will bind to the RNA region by Watson-Crick base pairing. Generally at least 10, for example at least 20, 40, 60, 80, 100, 200, 300, 400, 500 nucleotides of the antisense polynucleotide will be capable of binding to the RNA region by Watson-Crick base-pairing.

The antisense polynucleotide may be capable of hybridising to the RNA region under conditions of medium to high stringency such as 0.03M sodium chloride and 0.03M sodium citrate (or 0.03M sodium chloride and 0.003M sodium citrate) at from about 50 to about 60 degrees centigrade. Generally the antisense polynucleotide sequence has a degree of homology with the sequence of the RNA region (see below for further discussion of homology).

The polynucleotide may bind the full length viral RNA transcript and/or processed/spliced forms of the RNA. This binding leads to inhibition of viral replication, typically by inhibiting processing of the RNA, inhibiting the binding of the region to a viral or cellular factor or inhibiting the translation of the RNA.

Typically the antisense polynucleotide will inhibit IRV (e.g. strain HXBc2) replication in Jurkat cells. Generally the antisense polynucleotide is delivered by transfection or by retroviral (e.g. a MoMLV based) vector to the Jurkat cells.

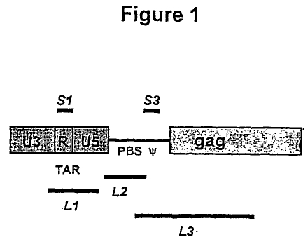

A preferred antisense polynucleotide binds the SDSV region of HIV RNA, but not the PBS or gag-encoding region. Thus in one embodiment the polynucleotide binds substantially only to the RNA region shown by S3 in FIG. 1.

Another preferred antisense polynucleotide binds at least the SD/V region and all or part of the gag encoding region of HIV-1 RNA, but not the U5 region. Typically the antisense polynucleotide binds at least 10, 20, 40, 100, 200, 300 or more nucleotides in the gag encoding region. In one embodiment the polynucleotide binds substantially only to the RNA region shown by L3 in FIG. 1.

A further preferred antisense polynucleotide binds at least the TAR region of HIV-1 RNA, but not the PBS region. Thus in one embodiment the polynucleotide binds substantially only to the RNA region shown by L1 in FIG. 1.

The antisense polynucleotide typically binds only to the sequence at positions

(i) 671 to 795, such as 681 to 785, preferably 691 to 775,

(ii) 640 to 1105, such as 650 to 1095, preferably 660 to 1085, or

(iii) 247 to 559, such as 257 to 549, preferably 267 to 539;

of HIV HXBc2 RNA, for example the HXBc2 sequence shown in Table III.

The antisense polynucleotide may bind within the specified sequences, either completely within (so that neither of the specified 5′ and 3′ positions are bound) or partially within (so that one of the specified 5′ and 3′ positions is bound).

The antisense polynucleotide may bind to a region of another HIV-1 virus corresponding to any of the specified regions above. The corresponding region is typically the same or has homology with the region of HIV HXc2 RNA (e.g. the region of the HXc2 sequence shown in Table III). Thus the corresponding region may be able to hybridise with the specified HIV HXBc2 regions (e.g. under the medium to high stringency conditions mentioned herein). The corresponding regions are typically capable of being amplified by PCR using the pairs of primers shown for S3, L3 and L1 in Table I.

The vector polynucleotide is capable of being expressed to produce the antisense polynucleotide in the cells of the host. Thus the polynucleotide typically also comprises control sequences which are operably linked to the sequence which is expressed to form the antisense polynucleotide, said control sequences being capable of expressing the expressed sequence in the cells of the host. Generally such cells are those which can naturally be infected by H-1, such as T cells, dendritic cells, monocytes (including macrophages) and epithelial cells (e.g. of the vaginal epithelium).

The control sequences are typically the same as, or substantially similar to, any of the control sequences in the gene of the host or of a virus capable of infecting the host, and in particular capable of infecting the cells mentioned above which can be infected by HIV-1. The control sequences typically comprise a promoter (generally 5′ to the expressed sequence) and/or a terminator and/or a polyadenylation signal and/or one or more enhancer sequences. The control sequences may cause constitutive expression. In a preferred embodiment the control sequences cause cell specific expression, which is typically specific for any of the cells mentioned above.

The vector polynucleotide may expressed transiently or stably. The polynucleotide may become integrated into the genome of the cell or may remain episomal. The vector may be in the form of a plasmid (typically circular) or artificial chromosome. The vector polynucleotide is generally at least 50, for example at least 500, 1000, 2000 nucleotides in length and up to at least 1 or more nucleotides in length.

The polynucleotide (including both the antisense and vector polynucleotide) may be in the form of a viral vector, typically based on a virus which is able to infect the host, and in particular any of the specific cells mentioned above. The vector is preferably derived from a retrovirus (e.g. lentivirus) vector. The virus vector is typically attenuated, and is, for example, replication defective.

In one embodiment the delivery of the polynucleotide is targeted, typically to any of the cells mentioned above or to infected cells. The polynucleotide may be associated with an agent which aids such targeting. Such an agent may comprise a receptor or ligand which binds to a ligand or receptor, respectively, expressed on the cells to be targeted. Such a receptor or ligand may be the natural receptor or ligand of the ligand or receptor to be targeted (or they may be a fragment and/or homologue thereof). Alternatively targeting may be achieved by an antibody, which typically binds a suitable ligand or receptor that is expressed on the cell to be targeted.

The polynucleotide itself may comprise a sequence that aids delivery of the polynucleotide to the cell. Such a sequence typically causes the polynucleotide to adopt a more compact form or aids its association with a targeting or transfection agent. The polynucleotide may be associated with transfection agent, a cationic agent (e.g. a cationic lipid), polylysine, a lipid or a precipitating agent (e.g. a calcium salt). Such agents generally aid the passage of the polynucleotide across the cell membrane. The polynucleotide may be in the form of liposomes or particles, for example in association with any of the agents mentioned herein. The particle typically has a diameter of 10 to 10⁻³ μm, for example 1 to 10⁻² μm. The polynucleotide may be in association with an agent that causes the polynucleotide to adopt a more compact form, such as a histone. The polynucleotide may be in association with spermidine.

The polynucleotide may be associated with a carrier which can be used to deliver the polynucleotide into the cell, or even into the nucleus, using ballistics techniques. Such a carrier may be a metal particle, such as a gold particle.

The polynucleotide may be in a substantially isolated form (e.g. in composition consisting essentially of the polynucleotide). The product may be mixed with carriers or diluents which will not interfere with the intended purpose of the product and still be regarded as substantially isolated. The polynucleotide may also be in a substantially purified form, in which case it will generally comprise at least 90%, e.g. at least 95%, 98% or 99% of the polynucleotide or dry mass of the preparation. The polynucleotide may be in the form of ‘naked DNA’.

The polynucleotide may be chemically modified, typically to enhance resistance to nucleases or to enhance its ability to enter cells. For example, phosphorothioate nucleotides may be used. Other nucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. The nucleic acids may be LNA's (locked nucleic acids), for example conformationally constrained by a 2′-O, 4′-C-methylene bridge. Alternatively mixed backbone oligonucleotides (MBOs) maybe used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nuclease's or 2′-O-alkyloligoribonucleotides. The polynucleotide may be a PNA (peptide nucleic acid).

The invention also provides a product which binds the same region of HIV-1 RNA as is bound by the antisense polynucleotide for use in treating or preventing HIV-1 infection. Thus the product may have HIV-1 RNA binding characteristics which are similar to any of the antisense polynucleotides mentioned, i.e. binding the same portions of the RNA and not binding other portions which are not bound by the antisense polynucleotide.

Thus a preferred product binds the SD/ψ region of HIV RNA, but not the PBS or gag encoding region. In one embodiment the product binds substantially only to the RNA region shown by S3 in FIG. 1.

In another preferred embodiment the product binds at least the SDST region and all or part of the gag encoding region of HUV-1 RNA, but not the U5 region. Typically the product binds at least 10, 20, 40, 100, 200, 300 or more polynucleotides in the gag encoding region. In one embodiment the product binds substantially only to the RNA region shown by L3 in FIG. 1.

In a further preferred embodiment the product binds at least the TAR region of HIV-1 RNA, but not the PBS region. Thus in one embodiment the product binds substantially only to the RNA region shown by L1 in FIG. 1.

The product typically binds only to the sequence at positions

(i) 671 to 795, such as 681 to 785, preferably 691 to 775, or

(ii) 640 to 1105, such as 650 to 1095, preferably 660 to 1085, or

(iii) 247 to 559, such as 257 to 549, preferably 267 to 539;

of HIV HXBc2 RNA (for example the HXBc2 sequence shown in Table III) or only to the corresponding sequence of the RNA of another HIV-1 virus.

The product typically is or comprises a polypeptide, a polynucleotide (for example a ribozyme or aptamer) or an organic molecule. It may be a naturally occurring or non-naturally occurring molecule.

The invention also provides particular fragments of an HIV-1 RNA which may or may not be part of the nucleotide sequence of a larger polynucleotide, which larger polynucleotide is not a naturally occurring HIV-1 RNA molecule. Thus typically the fragments will not comprise any further nucleotide sequence to their 5′ or 3′ or will only be flanked by sequence which is not present to their 5′ or 3′ in the HIV-1 RNA molecule from which they derive. In one embodiment the fragments are flanked by non-HIV sequence to their 5′ and/or 3′.

The fragments comprise sequence which is targeted by the antisense polynucleotides described above and preferably do not comprise sequence which is referred to as not being bound by the antisense polynucleotides. The fragments of the invention comprise:

(i) the SD/v region of HIV RNA, but not the PBS or gag encoding region; or

(ii) the SD/T region and all or part of the gag encoding region of HIV-1 RNA (generally at least 10, 20, 40, 100, 200, 300 or more nucleotides in the gag encoding region), but not the U5 region, or

(iii) the TAR region of HIV-1 RNA, but not the PBS region.

In one embodiment the fragments comprises or consists substantially only of the RNA region shown by S3, L3 or L1 in FIG. 1.

In one embodiment the fragment typically comprises or consists of any of the sequences defined by the position numbers below or sequence from within any of these defined sequences, which sequence has a length of at least 15, 20, 30, 50, 100, 200 or more nucleotides:

(i) 671 to 795, such as 681 to 785, preferably 691 to 775,

(ii) 640 to 1105, such as 650 to 1095, preferably 660 to 1085, or

(iii) 247 to 559, such as 257 to 549, preferably 267 to 539;

of HIV HXBc2 RNA (for example the Hxc2 sequence shown in Table III) or the corresponding sequence of another HIV-1 virus.

The fragment generally has a length of at least 15 nucleotides, such as at least 30, 50, 100, 200 or more nucleotides, for example up to a maximum of 500 nucleotides.

The invention also provides a method of identifying a product that is capable of treating or preventing HIV-1 infection comprising determining whether a candidate substance is capable of targeting any of the above defined specific regions of HIV-1 RNA which are targeted by the antisense polynucleotide. The targeting will comprise binding the region in a sequence specific manner and optionally acting on it.

In one embodiment of the method the candidate substance is contacted with the specific region to be targeted (and substantially no other region) to determine whether or not the substance binds or acts on the specific region. Typically in the method only a fragment of HIV-1 RNA is present such as any of the fragments mentioned herein.

In another embodiment of the method the substance is contacted with any naturally occurring HIV-1 RNA, such as the whole genome, and it is determined whether or not the substance binds to the specific region (and does not bind to other regions).

Binding may be detected by using any suitable means in the art. It may be detected by measuring the presence of the complex formed between the substance and HIV-1 RNA, for example in a ‘band shift’ which measure whether the presence of the candidate substance alters the mobility (typically retarding mobility) of the RNA in gel electrophoresis. Alternatively binding may be measured in a competitive binding assay in which whether or not the presence of the candidate substance reduces the binding between the HIV-1 RNA and a compound known to bind the RNA.

In the method whether or not the substance acts on the region may also be determined, for example whether or not the product cleaves the RNA in the region.

The product identified in the method is typically tested further to determine whether it is effective in treating or preventing HIV-1 infection in cellular assays or in vivo. It may also be tested to ensure that it is not toxic to humans.

Administration

The polynucleotide or product (including the product identified in the method of the invention) may be administered to a human or animal host at risk of HIV-1 infection or in need of treatment (due to having an HIV-1 infection). The likelihood of the host becoming infected is thus decreased or the condition of an infected host can be improved.

The polynucleotide or product may combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, vaginal, rectal, intravenous, subcutaneous, or transdermal administration.

The dose administered to a patient will depend upon a variety of factors such as the age, weight and general condition of the patient, the stage which the infection has reached, and the particular polynucleotide or product that is being administered. A suitable dose may however be from 0.1 to 100 mg/kg body weight such as 1 to 40 mg/kg body weight.

The polynucleotide (or products which are polynucleotides) may be administered directly as a naked nucleic acid construct. Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™).

When the polynucleotide of the invention is delivered in the form of a viral vector, the amount of virus administered is typically in the range of from 10⁶ to 10¹¹ infectious units/ml, preferably from 10⁷ to 10⁹ infectious units/ml. When injected, typically 1-2 ml of virus in a pharmaceutically acceptable suitable carrier or diluent is administered. The routes of administration and dosages described above are intended only as guide since a skilled physician will be able to determine readily the optimum route of administration and dosage for any particular patient and condition.

Homologues

Polynucleotides which have homology with another polynucleotide (e.g to viral RNA region) are referred to herein. When examining the homology between an antisense sequence and its target it will of course be necessary to compare a sequence which is the (exact) complement of the antisense sequence with the target sequence.

Typically a polynucleotide which is homologous to another polynucleotide is at least 70% homologous to the polynucleotide, preferably at least 80 or 90% and more preferably at least 95%, 97% or 99% homologous thereto. Such homology may exist over a region of at least 15, preferably at least 30, for instance at least 40, 60 or 100 or more contiguous nucleotides.

Methods of measuring polynucleotide homology are well known in the art. For example the UWGCG Package (Devereux et al (1984) Nucleic Acids Research 12, 387-395) provides the BESTFIT program which can be used to calculate homology (for example used on its default settings). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see. e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous polynucleotide typically differs from the original sequence by substitution, insertion and/or deletion, for example by at least 2, 5, 10, 20, 50 or more substitutions, deletions and/or insertions of nucleotides.

The invention is illustrated by the Examples:

### EXAMPLE 1

Aims and Results

The purpose of this study was first, to identify antisense sequences complementary to these regions in HIV-1 with optimal inhibitory characteristics, when expressed both by transfected DNA, and by transduced retroviral vectors in CD4+ T lymphocytes. We also attempted to identify the predominant mode of inhibition of viral replication caused by these molecules. In this paper we demonstrate significant inhibition of HIV-1 replication by particular antisense polynucleotides. Such polynucleotides target the TAR and SD/ψ regions, and may inhibit encapsidation and translation of viral RNA.

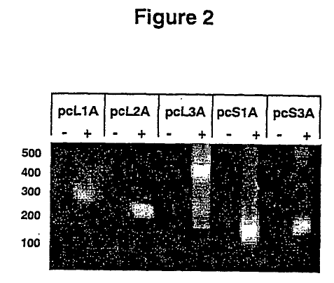

Of the three target regions within the HIV-1 5′ leader and LTR (TAR, PBS and ψ/SD) one long antisense sequence was designed to target the PBS, and long and short antisense sequences were designed to target TAR and the ψ/SD region (FIG. 1); To achieve high level expression of antisense RNA in lymphocytes an expression vector, pcDNA3.1, utilising the CMV immediate early promoter was chosen. Once these sequences, designated S1, L1, L2, S3 and L3, were cloned into pcDNA3.1 in both the sense and antisense orientations, each construct, along with pcDNA3.1, was stably transfected into Jurkat T cells. To demonstrate expression of antisense RNA sequences in T lymphocytes cellular RNA from G418-resistant cell lines was extracted and probed by RT-PCR FIG. 2 shows the results of RT-PCRs performed on each of the cell lines expressing antisense RNA along with negative control reactions, indicating RNA of the appropriate size could be detected in every cell line.

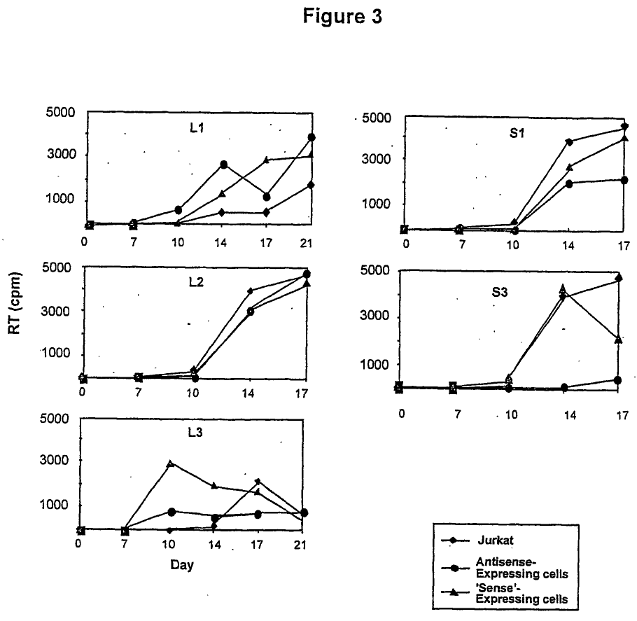

To assess potential resistance of cell lines expressing antisense RNA to challenge with HV-1, cell lines stably expressing pcDNA3.1 constructs were challenged with between 10⁴ and 10⁶ TCID50/ml of IIIB virus and subsequent viral replication measured by RT assays. FIG. 3 shows the results of challenge assays for all of the cell lines generated illustrating that only the cells expressing pcS3A showed significant resistance to HIV-1 replication as compared to control cell lines, although a minor degree of inhibition was noted with L3A. In the case of pcS3 A, cell lines also showed substantial resistance up to Day 14 at a challenge dose of 106 TCID50/ml. To confirm that resistance to viral challenge was not caused by reduced levels of surface CD4 in these cell lines, CD4 expression was measured by FACS analysis. The expression was found to be similar to control cells (data not shown) confirming that resistance to replication could not be attributed to reduced viral entry via CD4.

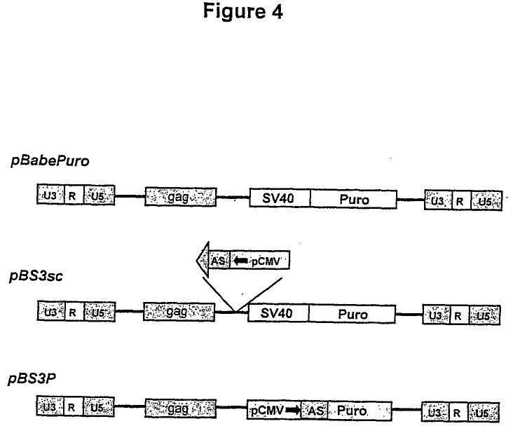

A prerequisite to designing antiviral vectors for HIV gene therapy is the ability to deliver antiviral genes to primary T lymphocytes, and recombinant retroviral vectors remain one of the most efficient vehicles for this purpose. A MoMLV-based vector, pBabePuro¹, was used to produce two different constructs expressing the L3, S3 and L1 sequences as these showed more effect when stably expressed in transfected cells, either as a single-copy cassette (e.g. pBS3sc) or as a fusion transcript containing, in addition, the puromycin resistance gene (e.g. pBS3P): see FIG. 4. The latter construct was designed with the intention of increasing both the expression of antisense RNA in target cells and also vector titres of retroviral vector particles through higher expression of vector RNA in producer cell lines. The FLY-A13 producer cell line utilising a MLV amphotropic envelope expressor and MoMLV gag-pol expressor³, was used to produce replication-incompetent retroviral particles for transducing Jurkat cells in the first instance. When cell lines stably expressing these constructs were generated in FLY-A13 cells, except for two cell lines, vector titres of only 10³ to 10⁴ cfu/ml were obtained: substantially lower than those originally obtained with this cell line³. Only cells expressing pBS3sc and pBabePuro itself generated titres higher than this at 5.8×10⁶ and 1.3×10⁵ cfu/ml respectively. Plasmids pBS3sc and pBabePuro, were successfully transfected into Jurkat cells to provide control cells for subsequent viral challenge experiments.

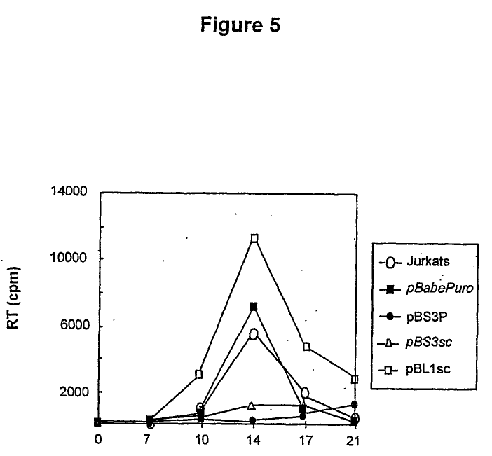

Jurkat cells either transduced or transfected with the pBabePuro constructs were challenged with IIIB at identical challenge doses as previously (FIG. 5). The cell line transduced with a vector expressing antisense RNA, pBS3sc, showed profound resistance to replication of HIV-1. Stable transfection with the construct pBS3P also showed inhibition of replication on viral challenge. There was no significant resistance seen in cells expressing either the L1 or L3 sequences from pBabePuro constructs (data not shown). Thus the S3 sequence consistently inhibited viral replication when expressed in T lymphocytes in a variety of different vectors including transduced or transfected murine retroviral vectors.

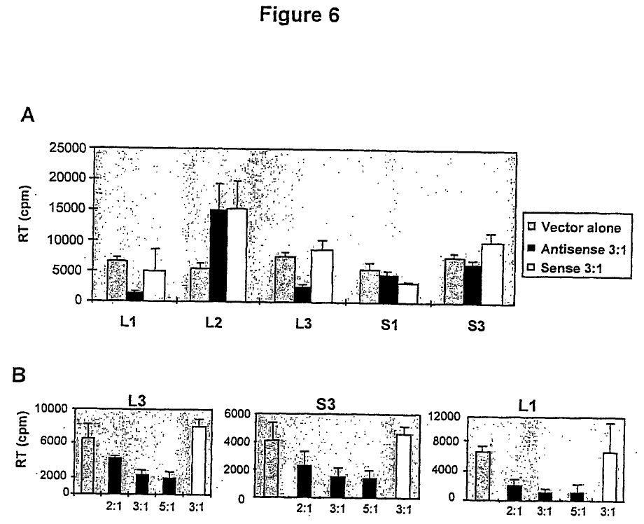

We performed, in addition, assays where constructs expressing effector sequences in either the ‘sense’ or antisense orientations, along with pcDNA3.1, were co-transfected with an HIV-1 Gag-Pol expressing construct, LψGPH, into COS-1 cells. This was for two reasons. Firstly, suppression of Gag-Pol production in cells co-transfected with antisense constructs would provide supportive evidence of an inhibitory effect of these RNA sequences, in addition to viral challenge assays. Secondly, analysis of cytoplasmic and virion RNA from cells transfected with these constructs might provide insights into the site of action of antisense RNA sequences in the transcription-translation pathways (and subsequent steps such as RNA packaging in the case of some of the antisense sequences). In the first instance evidence of suppression of Gag-Pol production by expression of antisense RNA was sought by measuring levels of reverse transcriptase in cell culture supernatants 48 hours after co-transfection of plasmids. FIG. 6 illustrates the results of the co-transfection assays, showing that, at a 3:1 ratio (antisense:vector DNA), two of the five antisense constructs, L1 and L3, and to a lesser extent S3 inhibited Gag-Pol production (FIG. 6A). When these constructs were co-transfected at variable ratios to the vector construct (FIG. 6B), both L1 and L3 showed a dose-response relationship between the degree of inhibition and amount of antisense-expressing plasmid transfected. There was a similar, albeit less dramatic trend for S3. These results imply a significant inhibitory effect of the S3, L1 and L3 antisense RNA sequences on the expression of Gag-Pol protein from transfected vector DNA, presumably directly or indirectly due to their antisense action. Assuming the level of RNA transcribed is proportional to the amount of plasmid transfected, the results of co-transfection assays suggest a dose-response for L1 and L3 and not S3 and imply that the inhibitory effect of this latter molecule is maximal at a relatively low level of RNA expression.

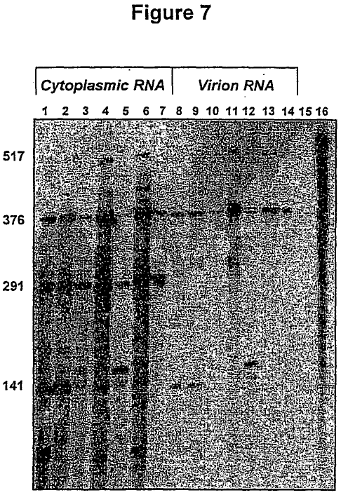

Analysis of cytoplasmic and virion RNA from a representative co-transfection experiment by ribonuclease protection assay (RPA) is illustrated in FIG. 7 and Table 2. Although there is a disparity between levels of genomic RNA signal between different control samples (despite equal quantities of cytoplasmic RNA being probed and then loaded) it is still possible to make assessments of the effects of antisense RNA expression on the relative levels of cytoplasmic and virion vector RNA. Relative to ‘sense’ constructs, there is a striking reduction in signal intensity of genomic RNA from cells expressing each of the antisense RNA sequences, particularly pcL3A and pcS3A. For L1A and L3A this is complemented by a further decrease of encapsidated RNA whereas for S3A, despite the fall in genomic RNA and the fall in RT, the packaging efficiency is, if anything, higher.

The apparent decline in cellular RNA might be complicated by antisense RNA complexing with vector RNA, preventing probe binding, as opposed to destroying target RNA, and might be an explanation for this finding. There was also a notable reduction in the intensity of spliced RNA bands for antisense constructs compared to sense, although relative to the amount of genomic RNA, the reductions in spliced RNA do not appear to be significant. Therefore whilst it is possible to infer that expression of each of these antisense molecules led to reduced levels of cytoplasmic genomic RNA, it is much less clear whether these RNAs had any specific effect on splicing.

Since the antisense RNAs decrease viral particle production more than can be explained by falls in encapsidation and in some cases affect cellular levels of viral mRNA, a significant action of these antisense RNA molecules seems to occur at the level of mRNA processing leading to reduced levels of both genomic and spliced RNA being exported to or surviving in the cytoplasmic compartment. However, in addition, control cells co-transfected with pcL1A and pcL3A both yielded lower levels of virion RNA compared to both their ‘sense’ controls and ‘vector alone’ samples, suggesting additional specific inhibitory effects on encapsidation of genomic RNA.

Of the five antisense sequences expressed in Jurkat cells, one, S3, targeting the SD/ψ region, showed significant antiviral activity in all assays when expressed from both plasmid and retroviral vectors, as well as in a co-transfection assay. Its longer counterpart, L3, showed moderate activity in the initial challenge assays, however this effect was not repeated when expressed from within a retroviral vector. The superior antiviral effect of L1 compared to S1, in the co-transfection assay, may relate to additional targeting of the U3 region—present at the 3 end of all viral RNA transcripts, or possibly more effective inhibition of the Tat-TAR interaction. Another possible interpretation may simply be that longer sequences, when optimally expressed (in COS-1 cells), are more efficient at forming stable RNA-RNA duplexes leading to greater inactivation of target mRNAs.

These studies have raised several very important questions about antisense therapy. Firstly, it is important to perform different studies in different model systems in order to optimise the effect. Assessment by cotransfection alone would have given misleading results and might have made it less likely that we would have considered antisense targeting the leader region downstream of the splice donor which, in effect, proved to be a consistent inhibitor of viral replication. Secondly, these experiments have been done at a level of viral challenge which is considerably higher than those used in other studies. We wished to give our therapeutic molecules the most stringent test available and it is clear that the S3 antisense is capable of providing inhibition even at this very high viral challenge. Given that the particle to infectivity ratio of EV is something under 10⁴, the S3 antisense is clearly conferring a protective efficacy against an extremely and probably unphysiologically high concentration of infectious particles. Thirdly, we were frustrated and surprised by the low level of expression of all of the antisenses in the cells. They were virtually undetectable by RNase protection which means that they are being expressed at a significantly lower level than common housekeeping cellular messenger RNAs and HIV RNA which we can readily detect in infected cells. Despite this, the comparability of the RT-PCR suggests that the efficacy of the antisense is not purely a function of the level of expression. These findings might suggest that the antisense molecules are actually targeting other mRNAs or that they are exerting their effect in the cells in which they express at a particularly susceptible time/location of virus replication. This extraordinarily low expression together with high efficacy is paradoxically very promising information for future antisense clinical studies.

One important advantage of antisense RNA over antiviral genes expressing novel proteins is that expression of these genes is unlikely to lead to immune responses against cells containing these genes although immune responses against marker genes may be seen if they are present in the transduced construct. In addition, as demonstrated in this study by the efficacy of one antisense sequence less than 100 nucleotides in length, multiple antisense genes could be expressed by one transduced vector.

Whilst it might be argued that the co-transfection assays provide little useful information relevant to the physiological situation (where the ability of CD4+ cells expressing antisense RNA to resist viral replication after challenge is paramount), this assay permits a more rigorous delineation of antisense effects over a single round of ‘infection’, in addition to providing clues as to the mechanism of action of antisense RNA. The observation that each of the antisense sequences which had suppressed Gag-Pol production in the co-transfection assays reduced levels of both spliced and unspliced cytoplasmic RNA suggests a significant effect on viral RNA prior to translation, and is consistent with the postulated actions of antisense RNA in disrupting nuclear processing of target RNA and leading to degradation of target sequences by cellular enzymes.

It is difficult to draw any firm conclusions about the effect of antisense RNA on subsequent steps in the viral life cycle. If, however, the results suggesting that L1 and L3 specifically inhibit genomic RNA encapsidation are significant, one might conclude that blocking this particular stage of the life cycle requires a longer antisense sequence, possibly more capable of maintaining a stable RNA duplex than shorter sequences.

### EXAMPLE 2

Materials and Methods

Construction of Antisense-Expressing Vectors

Sequences from the HIV-1 molecular clone HXB2, designated S1-L3 (FIG. 1) were amplified by PCR using primers containing a HindIII site. The size and positions of these sequences along with the PCR primers used to amplify them are shown in Table 1. PCR products were digested with HindIII then ligated into the HindIII site of pcDNA3.1 (Invitrogen, The Netherlands). Recombinants containing these sequences both in the sense and antisense orientations were identified by restriction digestion and sequencing; these constructs were named pcS1A (antisense orientation) and pcS1S (sense orientation) etc. Antisense-expressing vector constructs based on pBabePuro¹ were constructed as follows (and are illustrated in FIG. 4).

For single-copy vectors, antisense cassettes containing L1, L3 and 53 from the pcDNA3.1-based constructs were excised from these plasmids by digestion with NruI and BamHI and cloned into pBabePuro (linearised with BamHI and SnaBI). Cassettes were removed from the pcDNA3.1 constructs by digestion with NruI and EcoRV including the CMV promoter but no polyA and ligated into the pBabePuro (linearised by NheI). The monocistronic vector, pBS3P, where S3 antisense RNA is expressed as the upstream part of a transcript containing the puromycin resistance (PuroR) gene from the CMV IE promoter (pCMV), was initially constructed by excision of the SV40 promoter from pBabePuro using BamHI and HindIII. The pCMV-antisense cassette from pcS3A was removed by digestion with BglII and EcoRV and cloned into pBabePuro. PCR reactions were performed as for the RT-PCR protocol.

Cell Culture, Transfection and Transduction of Vectors

Jurkat cells were maintained in RPMI-1640 medium containing penicillin-streptomycin and 10% foetal bovine serum (FBS). Cells were transfected by electroporation at 550 mV and 25 μF and selected with either G418 (1.5 mg/ml) or puromycin (0.5 μg/ml). Monolayer cells (FLY-A13, COS-1 and NIH 3T3) were grown in Dulbecco's modified eagle medium (DMEM) containing penicillin-streptomycin and supplemented with 10% FBS. COS-1 cells were transfected by the DEAE-dextran method as described previously². pBabePuro-based retroviral vectors were stably transfected into FLY-A13 cells (ATCC CCL81) using Fugene™ (Boehringer-Mannheim (Roche, East Sussex, UK)) according to the manufacturer's instructions, and once puromycin-resistant colonies were generated antibiotic selection was removed and supernatant from the cultures removed two days later. Supernatant was immediately applied either to NIH 3T3 cells (to determine vector titres) or Jurkat cells in the presence of polybrene at 5 μg/ml for four hours, the transduction repeated the following day and antibiotic selection applied 24 hours later. Jurkat and FLY-A13 cells were selected with puromycin at 0.5 μg/ml, and transduced NIH-3T3 cells selected at 1.5 μg/ml. The titre of retroviral vector-containing supernatant on NIH-3T3 cells was measured by serial dilution³.

### Antisense and Vector RNA Detection

Total cellular RNA was extracted from Jurkat cells using TRI reagent (Sigma) according to the manufacturer's instructions. Reverse transcriptase polymerase chain reactions (RT-PCR) were performed using 1 mg of RNA added to a reaction mix consisting of 4 μL 5× Reaction Buffer (Promega, (Southampton, UK)-250 mM Tris HCl pH 8.3, 375 mM KCl, 15 nM MgCl2, 50 mM DTT), 0.8 μl dNTPs (25 mM each), 0.4 μl primer (25 mM), 20 u RNasin (ribonuclease inhibitor—Promega) and 200 units MoMLV reverse transcriptase, made up to a final volume of 20 μl. The reaction mix was incubated at 37° C. for 1 hr, and the enzyme then inactivated at 95° C. for 10 min.

For each sample the reaction was performed in duplicate with one reaction not containing the RT enzyme (negative control). Non-transfected cells were also tested as controls (not shown). PCR was performed using a DNA thermal cycler (Perkin-Elmer Cetus). Reactions consisted of an initial denaturation at 94° C. for 5 minutes followed by 35 cycles of a denaturation step at 94° C. for 1 minute, an annealing step at 56° C. for 1 minute and an extension step at 72° C. for 1.5 minutes. A typical 50 μl reaction would contain a DNA template, 25 pM of each oligonucleotide primer, 200 mM dNTPs and 1 unit of T. aquaticus DNA polymerase (Taq; Bioline, UK) in 1×PCR buffer (Bioline—10 mM Tris-HCL—pH 8.4, 50 mM KCl, 2 mM MgCl2, 0.01% gelatin, 0.5% Tween-20, 0.1% Triton X-100) overlayed with mineral oil. DNA templates for PCR were either 10 ng of linearised plasmid DNA (positive control) or 5 μl of RT reaction mix products. The primer used for reverse transcription was complementary to the SP6 sequence downstream of the multi-cloning site in pcDNA3.1 (position 989-1010). The second, PCR, stage of the RT-PCR was performed using the same primers (Table 1) originally employed for amplifying the target sequences.

Cytoplasmic and virion RNA were extracted from COS-1 cells using the method as previously described⁴. For ribonuclease protection assays (RPA), reactions were performed using the Ambion (Austin, Tex., USA) RNase protection assay kit, according to the manufacturer's instructions. Viral particles were normalised by RT activity and cellular message was normalised for total cellular RNA as previously described⁴. The DNA template for synthesis of radiolabelled RNA probes, KSIIψCS⁴, was linearised with XbaI producing a 517-nucleotide HxB2-specific riboprobe capable of distinguishing unspliced from spliced HIV-1 transcripts, and transfected plasmid DNA on the basis of the size of protected fragments.

Challenge of Jurkat Cells with HIV-1 and RT Assays

Jurkat cells were challenged with HIV-1 (IIIB) virus stocks in a 96-well format in order to permit large numbers of individual challenges to be performed concurrently at different concentrations of input virus. 10⁴ cells in 200 μl media were challenged at doses of virus between 10⁴ and 10⁶ TCID50/ml. Typically each cell population was challenged at five different doses (4×10⁶, 106, 2.5×10⁵, 6×10⁴ and 1.5×10⁴ TCID50/ml) with 4 wells at each concentration. Media was replaced from cultures twice weekly, and from 7 days after challenge RT levels were calculated from each well twice weekly for three weeks. Reverse transcriptase assays were performed on 10 μl samples of cell culture supernatant (in viral challenge experiments), or 10 μl of PEG-precipitated supernatant preparation derived from 10 mls of supernatant resuspended in 100 μl of PBS (for COS-1 cell co-transfections). The method used for this assay was the mini-RT assay described by Steffens⁵. RT levels were quantitated on a Packard Beta Counter (Packard Bell).

Co-Transfection Assays in COS-1 Cells

Antisense constructs were co-transfected with an HIV-1 (IIIB) vector plasmid LWCGPH expressing all except the env open reading frame of the virus, LψGPH⁶, into COS-1 cells at approximately 70% confluence. Transfections were performed in duplicate in 10 cm dishes and 5 μg of LψGPH was transfected with either 10, 15 or 25 μg of antisense or sense-expressing constructs, or pcDNA3.1. 48 hours later supernatant was precipitated with polyethylene glycol (8000) and RT assays were performed. For each target sequence three separate experiments were performed where either pcDNA3, the antisense construct or the ‘sense’ construct were co-transfected with vector DNA (LψGPH).

Initially, the pcDNA3.1 constructs were co-transfected at a 3:1 ratio (in milligrams of plasmid) to LψGPK however where there appeared to be a significant inhibitory effect of an antisense construct, the experiments were repeated with additional ratios of 2:1 and 5:1 pcDNA3.1 constructs:LψGPH. In each of these variable ratio co-transfection experiments the total amount of transfected DNA containing the pCMV promoter was kept constant by supplementing antisense or sense constructs with pcDNA3.1. Results from three separate experiments were used to prepare data for mean RT levels for each co-transfection.

### REFERENCES

1. Morgenstern J P, Land H. Advanced mammalian gene transfer: high titre retroviral vectors with multiple drug selection markers and a complementary helper-free packaging cell line. Nucl. Acids Res. 1990; 18: 3587-3596.

2. Ausubel F M et al. 1991. Current protocols in molecular biology. Wiley-Interscience, New York.

3. Cosset F L et al. High-titer packaging cells producing recombinant retroviruses resistant to human serum. J. Virol. 1995; 69: 7430-7436.

4. Kaye J F, Lever A M L. Trans-acting proteins involved in RNA encapsidation and viral assembly inhuman immunodeficiency virus type 1. J. Virol. 1996; 70: 880-886.

5. Steffens D L, Gross R W. Sequencing of cloned DNA using bacteriophage lambda gt11 templates. Biotechniques. 1989; 7: 674-680.

6. Richardson J H et al. Helper virus-free transfer of human immunodeficiency vIr=s type 1 vectors. J. Gen. Virol. 1995; 76: 691-696.

TABLE I

Regions of HIV-1 HXB2 5′leader and LTR expressed as antisense RNA from
pcDNA3.1 and retroviral vectors, and primer sequences used for
amplification and RT-PCR detection of antisense sequences.

| | Size-bp | | |
|---|---|---|---|
| Sequence | (location) | Upstream Primer (5′-3′) | Downstream primer (5′-3′) |
| S1 | 56 (443-499) | GGAAGCTTGCCTGTACTGGG | ACCCTCGAGAGACCAGTTCGAAGG |
| L1 | 272 (267-539) | GGAAGCTTGACAGCCGCCTAG | TCGGAGTTATTTCGAACGG |
| L2 | 207 (531-738) | GGAAGCTTGCCTTGAGTGC | CCGCTCCCCGCCGCTTCGAAGG |
| S3 | 84 (691-775) | GGAAGCTTGGACTCGGCTTGCT | TGATCGCCTCCGTTCGAAGG |
| L3 | 425 (660-1085) | GGAAGCTTGCGAAAGGGAAACCA | TCTGTGGTTCCTTCGAAGG |

TABLE II
Relative concentrations of cytoplasmic and virion RNA from cells
co-transfected with HIV-1 vector plasmid and antisense constructs,
based on RPA gel illustrated in FIG. 7 (using NIH image)

| | Cytoplasmic RNA | | |
|---|---|---|---|
| | Genomic | Spliced | Virion RNA |
| Sample | (376 nt) | (291 nt) | (376 nt) |
| Vector | 1 | 3.2 | 1 |
| Alone | | | |
| pcL1S | 1.1 | 3.6 | 1.8 |
| pcL1A | 0.6 | 2.4 | 0.3 |
| pcL3S | 9.3 | 7.1 | 5 |
| pcL3A | 0.3 | 0.6 | 0.6 |
| pcS3S | 5.2 | 4.8 | 3.2 |
| pcS3A | 0.7 | 3.1 | 7.1 |

TABLE III
Sequence of HXBc2

| | |
|---|---|
| 1 | TGGAAGGGCT AATTCACTCC CAACGAAGAC AAGATATCCT TGATCTGTGG |
| 51 | ATCTACCACA CACAAGGCTA CTTCCCTGAT TAGCAGAACT ACACACCAGG |
| 101 | GCCAGGGATC AGATATCCAC TGACCTTTGG ATGGTGCTAC AAGCTAGTAC |
| 151 | CAGTTGAGCC AGAGAAGTTA GAAGAAGCCA ACAAAGGAGA GAACACCAGC |
| 201 | TTGTTACACC CTGTGAGCCT GCATGGAATG GATGACCCGG AGAGAGAAGT |
| 251 | GTTAGAGTGG AGGTTTGACA GCCGCCTAGC ATTTCATCAC ATGGCCCGAG |
| 301 | AGCTGCATCC GGAGTACTTC AAGAACTGCT GACATCGAGC TTGCTACAAG |
| 351 | GGACTTTCCG CTGGGGACTT TCCAGGGAGG CGTGGCCTGG GCGGGACTGG |
| 401 | GGAGTGGCGA GCCCTCAGAT CCTGCATATA AGCAGCTGCT TTTTGCCTGT |
| 451 | ACTGGGTCTC TCTGGTTAGA CCAGATCTGA GCCTGGGAGC TCTCTGGCTA |
| 501 | ACTAGGGAAC CCACTGCTTA AGCCTCAATA AAGCTTGCCT TGAGTGCTTC |
| 551 | AAGTAGTGTG TGCCCGTCTG TTGTGTGACT CTGGTAACTA GAGATCCCTC |
| 601 | AGACCCTTTT AGTCAGTGTG GAAAATCTCT AGCAGTGGCG CCCGAACAGG |
| 651 | GACCTGAAAG CGAAAGGGAA ACCAGAGGAG CTCTCTCGAC GCAGGACTCG |
| 701 | GCTTGCTGAA GCGCGCACGG CAAGAGGCGA GGGGCGGCGA CTGGTGAGTA |
| 751 | CGCCAAAAAT TTTGACTAGC GGAGGCTAGA AGGAGAGAGA TGGGTGCGAG |
| 801 | AGCGTCAGTA TTAAGCGGGG GAGAATTAGA TCGATGGGAA AAAATTCGGT |
| 851 | TAAGGCCAGG GGGAAAGAAA AAATATAAAT TAAAACATAT AGTATGGGCA |
| 901 | AGCAGGGAGC TAGAACGATT CGCAGTTAAT CCTGGCCTGT TAGAAACATC |
| 951 | AGAAGGCTGT AGACAAATAC TGGGACAGCT ACAACCATCC CTTCAGACAG |
| 1001 | GATCAGAAGA ACTTAGATCA TTATATAATA CAGTAGCAAC CCTCTATTGT |
| 1051 | GTGCATCAAA GGATAGAGAT AAAAGACACC AAGGAAGCTT TAGACAAGAT |
| 1101 | AGAGGAAGAG CAAAACAAAA GTAAGAAAAA AGCACAGCAA GCAGCAGCTG |
| 1151 | ACACAGGACA CAGCAATCAG GTCAGCCAAA ATTACCCTAT AGTGCAGAAC |

1. A polynucleotide which is
(i) an antisense polynucleotide that binds the splice-donor/packaging signal region (SD/v9) or the TAR region of HIV-1 RNA, or
(ii) a vector polynucleotide capable of expressing (i);
for use in a method of treating or preventing HIV infection.

2. A polynucleotide according to claim 1 which is in the form of a viral vector.

3. A polynucleotide according to claim 1 or 2 wherein the antisense polynucleotide binds the SD/ψ region of HIV-1 RNA, but not the PBS or gag encoding region.

4. A polynucleotide according to claim 1 or 2 wherein the antisense polynucleotide binds at least the SD/ψ region and all or part of the gag encoding region of HIV-1 RNA, but not the U5 region.

5. A polynucleotide according to claim 1 or 2 wherein the antisense polynucleotide binds at least the TAR region of HIV-1 RNA, but not the PBS region.

6. A polynucleotide according to claim 3; 4 or 5 wherein the antisense polynucleotide binds only to the sequence at positions 671 to 795, 691 to 775, 640 to 1105, 660 to 1085, 247 to 559 or 267 to 539 of HIV HXc2 RNA, or within the said sequences; or to the corresponding sequence of the RNA of another HMV-1 virus.

7. A polynucleotide as defined in any one of claims 3 to 6.

8. A product that binds the same region of HIV-1 RNA as is bound by the antisense polynucleotide of any one of claims 1 or 3 to 6; for use in a method of treating or preventing HIV-1 infection.

9. Use of a polynucleotide or product as defined in any one of the preceding claims in the manufacture of a medicament for preventing or treating HIV-1 infection.

10. A method of preventing or treating HIV-1 infection comprising administering an effective amount of a polynucleotide or product as defined in any one of claims 1 to 8.

11. A fragment of an HIV-1 RNA that comprises the SD/v region of HIV-1 RNA, but not the PBS or gag encoding region.

12. A fragment of an HIV-1 RNA that comprises at least the SD/T region and all or part of the gag encoding region of HIV-1 RNA, but not the U5 region.

13. A fragment of an EHV-1 RNA that comprises at least the TAR region of HIV-1 RNA, but not the PBS region.

14. A fragment of an HIV-1 RNA that comprises only the sequence at positions 671 to 795, 691 to 775, 640 to 1105, 660 to 1085, 247 to 559 or 267 to 539 of HIV HXBc2 RNA, or a sequence which has a length of at least 15 nucleotides from within any of these sequences; or the corresponding sequence of the RNA of another HIV-1 virus.

15. Method of identifying a product that is capable of treating or preventing HIV-1 infection comprising determining whether a candidate substance is capable of targeting a region defined in any of claims 1 or 3 to 6 as binding the antisense polynucleotide, the finding that the substance is capable of targeting the said region indicating that the substance is capable of treating or preventing HIV-1 infection.

16. Method according to claim 15 comprising contacting the candidate substance with a region defined in any one of claims 1 or 3 to 6 as binding the antisense polynucleotide and determining whether the candidate substance binds and/or acts on the region.

17. Method according to claim 16 wherein the region is in the form of a fragment as defined in any one of claims 11 to 14.

18. A product identified in a method according any one of claims 15 to 17.

19. Process of manufacturing a medicament comprising carrying out the method of any one of claims 15 to 17 and combining the product identified in the method with a pharmaceutically acceptable carrier or diluent.
